(19) United States
(12) Patent Application Publication
(10) Pub. No.: US 2018/0221538 A1
(43) Pub. Date: Aug. 9, 2018

(54) Metallic and/ or ceramic components with at least one osseointegrative and osteoinductive surface (multi)layer structure

(71) Applicant: CeramTec GmbH, Plochingen (DE)

(72) Inventors: Alessandro Alan PORPORATI, Esslingen (DE); Marita RASCHKE, Leonberg (DE); Robert STREICHER, Feusisberg (CH); Meinhard KUNTZ, Esslingen (DE); Roman PREUSS, Kirchheim unter Teck (DE); Lukas GOTTWIK, Bad Boll (DE)

(21) Appl. No.: 15/747,808

(22) PCT Filed: Jul. 28, 2016
(86) PCT No.: PCT/EP2016/067973

§ 371 Date: Jan. 26, 2018

(30) Foreign Application Priority Data
Jul. 28, 2015 (EP) 15178661.3

(51) Int. Cl. A61L 27/30; A61L 27/10

(52) U.S. Cl. CPC A61L 27/306; A61L 27/10; A61L 2430/02; A61L 2420/08; A61L 2420/02; A61L 2420/04

(57) ABSTRACT

The present invention relates to metallic and/or ceramic components, in particular in the field of medical technology, having improved osseointegrative and osteoinductive properties. The present invention also relates to a method for producing the ceramic components.

The present invention relates to metallic and/or ceramic components, in particular in the field of medical technology, having improved osseointegrative and osteoinductive properties, the metallic and/or ceramic components are suitable for a cementless fixation in the human body, for example the acetabular region of the human pelvis. The present invention also relates to a method for producing the metallic and/or ceramic components.

Nowadays metal shells with osseointegrative surface structures are necessary for a cementless fixation of ceramic inserts in the acetabular region of the human pelvis. One possibility to fix ceramic implants directly into bone is described for example in EP 1268364 A1, wherein a ceramic body is layered with a surface structure having open pores.

However, it was an object of the present invention to further enhance the osseointegrative properties, that is the ability to promote bone ingrowth, of metallic and/or ceramic components

This object is solved by the metallic and/or ceramic component of the present invention which comprises an osseointegrative and osteoinductive surface (multi)layer structure, wherein a surface layer of the surface (multi)layer structure comprises strontium aluminate according to claim 1. It also enables the direct implantation of the metallic and/or ceramic component due to the addition of the porous osseointegrative and osteoinductive surface (multi)layer structure comprising strontium aluminate to the backside of a ceramic substrate.

The metallic and/or ceramic component comprising the osseointegrative surface (multi)layer structure of the present invention is able to increase the bone growth rate due to the presence of strontium aluminate in the osseointegrative and osteoinductive based layer on the metallic or ceramic surface of the metallic and/or ceramic component. The surface (multi)layer structure may have also osteoinductive properties and therefore may promote the growth, maturation and activity of osteoblasts. Strontium is known as bone forming agent. Strontium-containing drugs are already used as systemic osteoblast-activating medication in various clinical settings promoting mechanical stability of the osteoporotic bone, but have not been described as an active ingredient for promoting bone ingrowth of ceramic components, in particular implants.

The term metallic and/or ceramic component as used in the present invention comprises components composed of metals only or ceramic materials only, but also components being mainly composed of ceramics and having a metallic surface for example. Preferred metallic and/or ceramic components of the present invention are ceramic components used in the field of medical technology.

As the metallic and/or ceramic component of the present invention is to be used in the field of medical technology, the component is preferably an implant, for example a cup in hip arthroplasty or a cage for the spine.

The ceramic component of the present invention comprises an osseointegrative and osteoinductive surface (multi)layer structure on its surface comprising at least one osseointegrative and osteoinductive layer. The osseointegrative surface structure is preferably a multilayer structure and comprises several osseointegrative layers, for example up to 30 layers. Preferably, the osseointegrative surface (multi)layer structure comprises from 1 to 5 osseointegrative layers.

The osseointegrative and osteoinductive surface (multi)layer structure of the present invention comprises a ceramic base material and strontium aluminate. The term strontium aluminate comprises any known strontium aluminate compound, such as the base strontium aluminate (SrAl₂O₄), monoclinic strontium aluminate (SrAl₄O₇), cubic strontium aluminate (Sr₃Al₂O₆), the hexagonal strontium aluminate (strontium hexaaluminate; SrAl₁₂O₁₉) and orthorhombic strontium aluminate (Sr₄Al₁₄O₂₆). The preferred compound is strontium hexaaluminate. The strontium aluminate is preferably present in platelet-form within the osseointegrative surface (multi)layer structure. The content of the strontium aluminate may be in the range of from 1 wt % to 50 wt %, preferably 1 wt % to 20 wt % based on the total weight of the surface (multi)layer structure.

The ceramic base material is selected from the group consisting of alumina based ceramics, zirconia based ceramics, preferably alumina based ceramics and zirconia based ceramics, more preferably a zirconia-platelet toughened alumina ceramic. The zirconia content in the zirconia-platelet toughened alumina ceramic is up to 25 wt % based on the total weight of the surface (multi)layer structure.

The osseointegrative and osteoinductive surface (multi)layer structure may exhibit a strontium gradient by means of different strontium contents in different layers of the osseointegrative surface (multi)layer structure with the aim to gradually release the residual stresses in the surface (multi)layer structure. In a preferred embodiment, the strontium content increases with the layer number, wherein the first/innermost layer has the lowest strontium content and the last/outermost layer has the largest strontium content. The outermost layer has to be seen as the layer coming in direct contact with the surrounding tissue or bone of the human body.

The amount of the strontium aluminate in the innermost layer of the respective strontium gradient structure is less than 5 wt %, preferably equal to or less than 1 wt % based on the total weight of the surface (multi)layer structure, with the proviso that the strontium content is not zero. The outermost layer comprises strontium aluminate in an amount of less than 25 wt %, preferably between 15 and 20 wt % based on the total weight of the surface (multi)layer structure.

The porosity of the osseointegrative and osteoinductive surface (multi)layer structure, that is the number of pores per unit volume, may be between 25% and 90%, preferably between 25% and 70%. The diameter of the pores of between is between 1 micron and 1000 microns, preferably between 20 microns and 600 microns. The number of pores per unit volume, their size, that is, its diameter, and its shape can advantageously be determined by the selection of a suitable pore-forming substance.

The thickness of the whole layer is between about 0.02 mm and 10 mm, preferably between 0.1 mm and 2 mm.

For the production of a ceramic component with an osseointegrative and osteoinductive surface multilayer structure, such as a highly osseointegrative and osteoinductive implant, a high content strontium alumina zirconia-based ceramic slurry can be deposited on a green substrate by using the method as described for example in EP 1268364 A1, where the substrate is formed as a green body of an inorganic material and to the substrate in the state of the green body, a suspension the same inorganic material constituting the substrate, or other material is applied. This suspension contains the layer material and additionally may contain a pore-forming substance or the pore-forming substance can also be applied separately. Only after applying the layer is a common heat treatment of substrate and layer by drying and sintering to produce a monolithic molding. The method for producing the substrate does not generally differ from those known from the prior art.

During the sintering, the green substrate achieves the full density; on the other hand the surface (multi)layer structure remains micro-porous, because of lower sintering activity caused by the high volume platelets content; and macro porous, because of pore forming or foaming agents. The diameter of the micro-pores is of between 0.05 micron to 5 micron, but preferably at submicron-level, from 0,1 micron to 1 micron, whilst the macro-pores have a diameter between 1 and 100 micron, preferably between 10 and 500 micron, with the proviso that the macro-pores are larger in diameter than the micro-pores. Moreover, it has to be understood that the micropores should look like “caves” into the material and the macropores like “valleys”.

The alumina-based ceramic slurry comprises alumina, zirconia, strontium oxide and yttrium oxide. During the sintering stage, the solid reaction between alumina and strontium oxide undergoes forming the strontium aluminate, in particular the strontium hexaaluminate.

In order to achieve a macro-porous osseointegrative and osteoinductive surface (multi)layer structure the respective ceramic slurry may also comprise (organic) additives such as pore forming agents or foaming agents based on the respective method for forming a porous layer. Such additives may be present in amounts of from 0.05 to 10 wt % based on the total weight of the surface multilayer structure composition. While the pore forming agent is pyrolized during the sintering process, the foaming agent is used in foaming process both leading to a porous osseointegrative and osteoinductive layer(s) having a porosity as disclosed above.

Thus, the porosity may be enhanced with organic pore-forming agents in the sintering process or with pore-forming agents or foaming agents and foaming procedures, respectively.

As pore-forming substances, in particular organic compounds, for example, starches, cellulose or waxes, and natural and synthetic polymers, which evaporate during the thermal treatment of the substrate and the deposited thereon layer gasify to consume or burn, thereby forming the pores are used.

The ceramic slurry may comprise further additives commonly used in the field of ceramic technology, such as binder.

Alternatively, it is also possible to apply the osseointegrative layer(s) on a substrate in sintered state. Thereby, the surface (multi)layer structure may be deposited on the sintered ceramic substrate or ad-hoc prepared metallic substrate by vapour- or plasma-deposition processes.

The osseointegrative and osteoinductive surface (multi)layer structure may be also grown on pre-existing macro-porous surface which might be ceramic foam or metallic osseointegrative surface. Micro- and macro-porosities are produced for an improved osseointegration and osteoinduction.

The osseointegrative and osteoinductive surface (multi)layer structure may also be deposited on structured surfaces of the ceramic component. A structured surface in the sense of the present invention is for example a porous surface. The structuring of the ceramic component surface may be produced by milling processes, turning processes or a combination of both. Another method for producing a structured surface is the method of ceramic injection molding. The structuring of the metallic or ceramic component surface may be produced by for example by additive manufacturing.

Alternatively, pore forming agents which are pyrolized during the sintering or foaming agents for foaming processes can be added to the substrate material in order to produce a structured surface.

Other methods for producing structured surfaces are grinding or electro discharge machining processes.

1. A ceramic component comprising an osseointegrative and osteoinductive surface (multi)layer structure on its surface comprising at least one osseointegrative layer wherein the osseointegrative and osteoinductive layer comprises strontium aluminate.

2. The ceramic component of claim 1, wherein the osseointegrative and osteoinductive surface (multi)layer structure comprises several osseointegrative and osteoinductive layers.

3. The ceramic component of claim 1, wherein the strontium aluminate is present in platelet-form.

4. The ceramic component of claim 2, wherein the osseointegrative and osteoinductive layers of the surface (multi)layer structure have different strontium contents.

5. The ceramic component of claim 4, wherein the strontium content increases with the layer number so that the outermost layer has the highest strontium content.

6. The ceramic component of claims 1, wherein the surface of the ceramic component is structured.

7. The ceramic component of claim 6, wherein the structuring of the surface is carried out by a process selected from the group consisting of a milling process, a turning process, a combination of a milling and a turning process, ceramic injection molding, a foaming process due to the addition of a foaming agent, a pore forming process during sintering due to the addition of a pore forming agent, a grinding process, electro discharge machining and additive manufacturing.

8. A method for producing a ceramic component in accordance with claims 1, wherein a ceramic slurry comprising strontium aluminate is applied to a substrate in green state.

9. A method for producing a ceramic component in accordance with claims 1, wherein the osseointegrative surface (multi)layer structure is applied to a substrate in sintered state.

10. The method of claim 9, wherein the osseointegrative surface (multi)layer structure is applied by vapor or plasma deposition.
